(19) United States
(12) Patent Application Publication
(10) Pub. No.: US 2010/0128839 A1
(43) Pub. Date: May 27, 2010

(54) Structure and Procedure for X-Ray CT Guided Cancer Treatment

(75) Inventors: Larry D. Partain, Los Altos, CA (US); Gary Virshup, Cupertino, CA (US); Edward Seppi, Portola Valley, CA (US); David Humber, Los Gatos, CA (US)

Correspondence Address: HOUST CONSULTING (Varian), P.O.BOX 2688, SARATOGA, CA, 95070-0688, US

(73) Assignee: Varian Medical Systems, Inc., Palo Alto, CA (US)

(21) Appl. No.: 12/275,819

(22) Filed: Nov. 21, 2008

(51) Int. Cl. A61N 5/10; H05G 1/60

(52) U.S. Cl. 378/4; 378/65

(57) ABSTRACT

A radiation apparatus includes a first radiation source configured to generate radiation suitable for therapeutic treatment, and a structure for supporting a body. The structure comprises a curved surface adapted to receive a body portion to be treated during a therapeutic treatment.

### BACKGROUND

This invention relates in general to radiation therapy and medical imaging, and in particular to improved structures and procedures for X-ray computed tomography (CT) guided cancer treatment.

Radiation therapy is one of the most effective modalities for the majority of cancers and with surgery, remains the most cost-effective way curing many cancers. Various radiation therapy systems and methods are known. For example, image-guided radiation therapy (IGRT) has been developed in which planar or volumetric imaging techniques are employed to measure target position and correct target positional errors immediately prior to or during treatment delivery. IGRT allows more accurate control of radiation delivery to a target such as a tumor while reducing exposure to the surrounding or adjacent healthy tissue or organs.

While achievements have been made in radiation therapy, challenges remain. For instance, conventional radiation therapy systems require couches with a flat surface for patient support and alignment. With a flat top, it is much easier to repeatedly position and align patients for treatment planning and delivery which can span over 6 weeks of daily treatment. However, as FIG. 1 illustrates, flat top support structures cause imaging artifacts, i.e., discrepancy between system value numbers in the reconstructed image and true attenuation coefficients of the object often quantified in Hounsfield units. FIG. 1 is a CT image of the pelvic region of a prostate cancer patient, which was acquired by an imaging system with the patient being supported on a couch with a flat surface 2. Artifacts in the form of streaks are apparent in FIG. 1 as indicated by the arrows 4.

Artifacts degrade the image quality, hide pathological areas, and sometime make the images diagnostically unusable. In cases where contrast agents are used to enhance the conspicuity of small cancer lesions, artifacts may lead to misidentification of healthy tissues as malignant.

### SUMMARY

The present invention provides radiation apparatuses and methods that can effectively avoid or mitigate artifact errors in computed tomography images which are otherwise introduced by conventional apparatus. In one embodiment, the radiation apparatus comprises a first radiation source configured to generate radiation suitable for therapeutic treatment delivery, and a structure for supporting a body. The first radiation source may also be suitable for screening, diagnosis, staging, treatment planning, positioning, targeting, and/or monitoring response in addition to treatment delivery. The structure comprises a curved surface adapted to receive a body portion to be treated during a therapeutic treatment. The radiation apparatus may comprise a first image acquisition device operatively disposed opposite to the first radiation source. The radiation apparatus may optionally comprise a second radiation source configured to generate radiation suitable for diagnostic imaging or other applications, and a second image acquisition device operatively disposed opposite to the second radiation source.

In some embodiments, the application includes the delivery of a therapeutic treatment, either alone or in combination with one or more of the following applications: (1) screening, (2) diagnosis, (3) staging, (4) treatment planning, (5) positioning, (6) targeting, and (7) monitoring response.

In some embodiments, the structure may comprise an elongate body having a first end, a second end, and a curved top surface extending from the first end to the second end. The structure may comprise a curved bottom surface extending from the first end to the second end. Alternatively, the structure may comprise a flat bottom surface extending from the first end to the second end.

The structure's curved surface may have a substantially continuous radius of curvature. In some embodiments, the curved surface may have a substantially constant radius of curvature.

In some embodiments, the structure may comprise an alignment line on the curved surface extending from the first end to the second end. The alignment line may be substantially centered on a symmetry axis of the curvature of the structure.

The structure may be removably coupled to the couch. The structure may be movable relative to the couch.

In some embodiments, the structure may have a curved surface forming a cup-shaped interior adapted to receive a body portion. The cup-shaped interior may be generally conformal to a shape of the body portion when pendulous.

In a preferred embodiment, a radiation apparatus comprises a first therapeutic radiation source, a first image acquisition device operatively disposed opposite to the first therapeutic radiation source, a second diagnostic radiation source, a second image acquisition device operatively disposed opposite to the second diagnostic radiation device, and a structure for supporting a body. The structure comprises a curved surface adapted to receive a body portion to be irradiated by radiation beams from the first therapeutic radiation source and the second diagnostic radiation source.

In one aspect, a method of irradiating a body portion of a patient comprises the following steps. A patient is placed on a structure. The structure comprises an elongate body having a first end, a second end, and a curved top surface extending from the first end to the second end and adapted to receive the patient. The elongate body comprises an alignment line over the curved surface. The patient is positioned in a laying position with reference to the alignment line. Radiation from a radiation source is delivered to a body portion of the patient after the patient is properly positioned. During the positioning step, one or more anatomies of the patient may be aligned with respect to the alignment line. Such anatomies may include navel, sternum, throat, nose, and eye etc. A light line may be optionally projected over the patient superposing the alignment line.

### BRIEF DESCRIPTION OF THE DRAWINGS

These and various other features and advantages will become better understood upon reading of the following detailed description in conjunction with the accompanying drawings and the appended claims provided below, where:

FIG. 1 is a computed tomography image of the pelvic region of a patient showing streak artifacts;

FIG. 2 is a schematic showing a radiation apparatus in accordance with some embodiments;

FIG. 3 is a schematic showing a structure comprising a curved top surface with a constant radius of curvature in accordance with some embodiments;

FIG. 4A is a schematic showing a structure comprising a curved top surface and a flat bottom surface in accordance with some embodiments;

FIG. 4B is a schematic showing a structure comprising angled side surfaces in accordance with some embodiments;

FIG. 4C is a schematic showing a structure comprising curved side surfaces in accordance with some embodiments;

FIGS. 5A and 5B are schematics showing an alignment line on a curved surface of a structure in accordance with some embodiments;

FIGS. 6A, 6B and 6C are computed tomography images of a pendulous breast supported by a structure having a flat surface; and

FIG. 7 is a computed tomography image of a pendulous breast supported by a structure having a cup-shaped cavity.

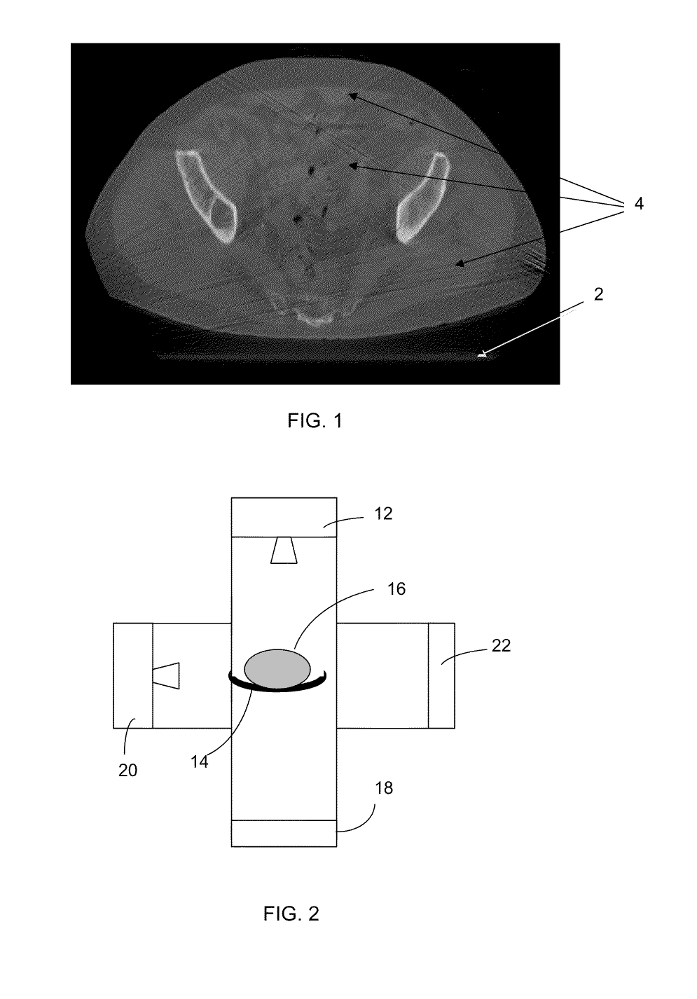

### DETAILED DESCRIPTION OF VARIOUS EMBODIMENTS

Various embodiments of apparatus and methods for radiation therapy and imaging are described. It is to be understood that the invention is not limited to the particular embodiments described as such may, of course, vary. An aspect described in conjunction with a particular embodiment is not necessarily limited to that embodiment and can be practiced in any other embodiments. It is also to be understood that the terminology used herein is for the purpose of describing particular embodiments only, and is not intended to be limiting since the scope of the invention will be limited only by the appended claims, along with the full scope of equivalents to which such claims are entitled.

In addition, various embodiments are described with reference to the figures. It should be noted that the figures are not drawn to scale, and are only intended to facilitate the description of specific embodiments. They are not intended as an exhaustive description or as a limitation on the scope of the invention.

All technical and scientific terms used herein have the same meaning as commonly understood by one of ordinary skill in the art to which this invention belongs, unless defined otherwise. As used in the description and appended claims, the singular forms of “a,” “an,” and “the” include plural references unless the context clearly dictates otherwise. Thus, for example, reference to “a radiation source” includes one or a plurality of sources, reference to “an image acquisition device” includes one or more of such, and reference to “the curved surface” includes one or more surfaces of the form or configuration described herein and equivalents thereof.

As used herein the term “flat surface” refers to a surface that is planar. The term “curved surface” refers to a surface comprising at least a portion that is curved or bent.

As used herein the term “vertical” refers to a direction aligned with the direction of the force of gravity. The term “horizontal” refers to a direction perpendicular to a vertical direction as defined above. Relative terms such as “on,” “upper,” “over,” “under,” “top,” “bottom,” “higher,” and “lower,” are defined with respect to the conventional plane or surface being on the top surface of the structure, regardless of the orientation of the structure, and do not necessarily represent an orientation used during manufacture or use. The following detailed description is, therefore, not to be taken in a limiting sense.

Referring to FIG. 2, a radiation apparatus 10 is described. In general, the radiation apparatus 10 comprises a first radiation source 12 adapted to generate radiation beams suitable for therapeutic treatment, and a structure 14 for supporting a body or body portion 16. The structure 14 comprises a curved surface adapted to receive a body portion to be treated during radiation therapy. The beams generated by the first radiation source 12 may also be suitable for imaging. Preferably the apparatus 10 comprises a first image acquisition device 18 operatively disposed opposite to the first radiation source 12. In addition, the apparatus 10 may optionally comprise a second radiation source 20 adapted to generate radiation beams suitable for diagnostic imaging, and a second image acquisition device 22 operatively disposed opposite to the second radiation source 20.

The first radiation source 12 is configured to generate radiation beams suitable for therapeutic treatment. Depending on the type, size, location of the target or other factors, the first radiation source 12 may be configured to generate radiation beams having energy levels at either megavoltages (MV) or kilovoltages (KV). As used herein, energy levels are expressed in terms of the electric potential used by a radiation source such as an accelerator or X-ray tube to produce e.g. photon beams. For example, in some embodiments, it would be desirable to employ X-ray tubes as the first radiation source 12 to generate beams with energy levels ranging from about 120 KV to about 1 MV, or preferably from about 200 KV-300 KV for therapeutic treatment. This would provide a good combination of low skin dose with moderate X-ray shielding requirements. In some embodiments, it would be desirable to employ accelerators as the first radiation source 12 to generate beams with energy levels ranging from about 900 KV to about 6 MV or higher for therapeutic treatment. In some embodiments, the radiation beams generated by the first radiation source 12 may also be used for imaging, e.g., to obtain images suitable for positioning a body portion. For example, an accelerator may be used as the first radiation source 12 to generate beams for both therapeutic treatment and imaging. An accelerator may be operated at up to 1 MV or higher to generate beams for obtaining lower contrast images suitable for positioning. Alternatively, an X-ray tube may be operated at low voltages e.g. from about 30 KV to about 80 KV, or preferably from about 50 KV to about 80 KV to generate X-ray beams suitable for diagnostic imaging. Both imaging and treatment can be done at megavoltages with increased image contrast resolution as the megavoltage values approach the 900 KV to 2 MV range. Both imaging and treatment can be done at kilovoltages using a single source and detector with the imaging performed at 80 to 120 KV and the tube voltage on the same high power X-ray tube increased to 200 to 400 KV for this treatment part.

The second radiation source 20 is configured to generate radiation beams suitable for diagnostic imaging. For diagnostic imaging of soft tissue e.g. breasts, it would be desirable in some embodiments to use beams produced by e.g. an X-ray source operating at reduced voltages such as from 30 KV upwards, or preferably from about 50 KV to about 80 KV. In some embodiments where metal surgical clips or implanted gold seeds etc. are used as fiducial markers for e.g. breast positioning, radiation beams with energy levels ranging from about 30 KV to multi-MV may also be used for imaging. U.S. Pat. No. 6,888,919 describes an X-ray radiation source that is capable of generating X-rays at different energy levels, the disclosure of which is incorporated herein by reference in its entirety.

A beam adjuster (not shown) may be positioned in front of the first and/or second radiation sources 12, 20 to adjust the shape, size, intensity, and direction of radiation beams. In one embodiment, the beam adjuster may include one or more multiple leaf collimators. In another embodiment, the beam adjuster may include one or more multiple leaf collimators and one or more single jaw collimators. In a preferred embodiment, the first and/or second radiation sources 12, 20, and the beam adjusters are configured to produce cone beams.

In some embodiments, the first and second radiation sources 12, 20 may be coupled to a common structure such as a gantry, and rotate or move together. Alternatively, the first and second radiation sources 12, 20 may be coupled to separate structures, such as gantries, C-arms or the like, and move or rotate separately. The two or more radiation sources 12, 20 may be located in close proximity with each other or separated from each other by e.g. 45 or 90 degrees. For example, in a preferred embodiment, two radiation sources may be so located that they project radiation beams toward a target at an angle of approximately 90 degree from each other. By way of example, U.S. Pat. No. 6,888,919 assigned to Varian Medical Systems, Inc., discloses a system including two or more X-ray radiation sources with different configurations and different energy levels. U.S. Pat. No. 6,888,919 is incorporated herein by reference in its entirety.

The first image acquisition device or imager 18 is configured to acquire image data sets generated by the radiation beams from the first radiation source 12 passing through the body portion 16. The second image acquisition device or imager 22 is configured to acquire image data sets generated by the radiation beams from the second radiation source 20 passing through the body portion 16. The first and second imagers 18, 22 are operatively disposed opposite to the first and second radiation sources 12, 20 respectively. Means such as robotic arms or the like may be used to position the first and/or second imagers 18, 22 opposite to the first and/or second radiation sources 12, 20 when in use, or retract the first and/or second imagers 18, 22 out of the way when not in use. The second diagnostic imager 22 may operate in a plane orthogonal to the therapeutic radiation beam from the first radiation source 12. The first and second imagers 18, 22 may operate in concert.

In some embodiments, the first and/or second imagers 18, 22 are configured to acquire cone beam image data sets. In a preferred embodiment, the first and/or second imagers 18, 22 may be flat plate imagers. In one embodiment, the first imager 18 may be a flat plate imager configured to acquire image data sets generated by cone radiation beams at MV energy levels. In some embodiments, the second imager 22 may be a flat plate imager configured to acquire image data sets generated by cone radiation beams at KV energy levels. In another embodiment, the first and/or second imagers 18, 22 are configured to acquire image data sets generated from radiation beams at multiple energy levels, such as at both MV energy levels and KV energy levels. U.S. Pat. No. 6,800,858, assigned to the same assignee, discloses X-ray image detecting devices that are capable of detecting multiple energy level X-ray images and can be used as image acquisition devices in accordance with the present invention. U.S. Pat. No. 6,800,858 is incorporated herein by reference in its entirety. It should be noted that the radiation apparatus 10 is not limited to X-ray radiation therapy and imaging. Depending on the nature of treatment or application, the first and/or second radiation sources 12, 18 may generate X-ray radiation or other kinds of radiation beams, which include, but are not limited to, electron ray beams, positron beams, proton beams, antiproton beams, neutron beams, heavy ion beams, e.g., alpha ray beams, carbon ion beams, etc. The first and/or second imagers 18, 22 may include different kinds of radiation sensors corresponding to different radiation beam sources.

The structure 14 supports a body or a body portion 16 in a position for radiation treatment and/or imaging. The structure 14 can perform one or more of multiple functions in addition to supporting a body or body portion. For example, the structure 14 may function to position and/or immobilize the body 16, physically protect the body from moving parts such as a radiation source, or be configured to reduce artifacts in the reconstructed images.

Artifacts in computed tomography (CT) refer to discrepancy between the CT numbers in the reconstructed image and the true attenuation coefficients of the anatomy. Artifacts may originate from a range of sources. For example, artifacts occur when a portion of a region of interest is outside of the scan field of view. The incomplete information relating to that portion leads to streaking or shading artifacts. Artifacts also occur when a region of interest has higher attenuation coefficients or scattering than its surroundings. The rapid changes of densities in the structures cause streaking artifacts and cupping artifacts. Artifacts degrade the quality of the reconstructed images and may cause misdiagnosis. The structure provided by this invention can effectively avoid or mitigate artifact errors in CT images which are otherwise introduced by conventional apparatus.

In some embodiments, the structure 14 preferably has a curved top surface adapted to receive a body portion 16 to be treated. For example, the structure 14 may have a top surface with a concave curve as viewed from a cross section.

In some embodiments, the structure 14 may be in an elongate form extending from a first end to a second end. A curved top surface may extend the entire length or a portion of the length of the structure. The length and the width of the elongate structure may be sufficient to receive all or at least a portion of a patient body.

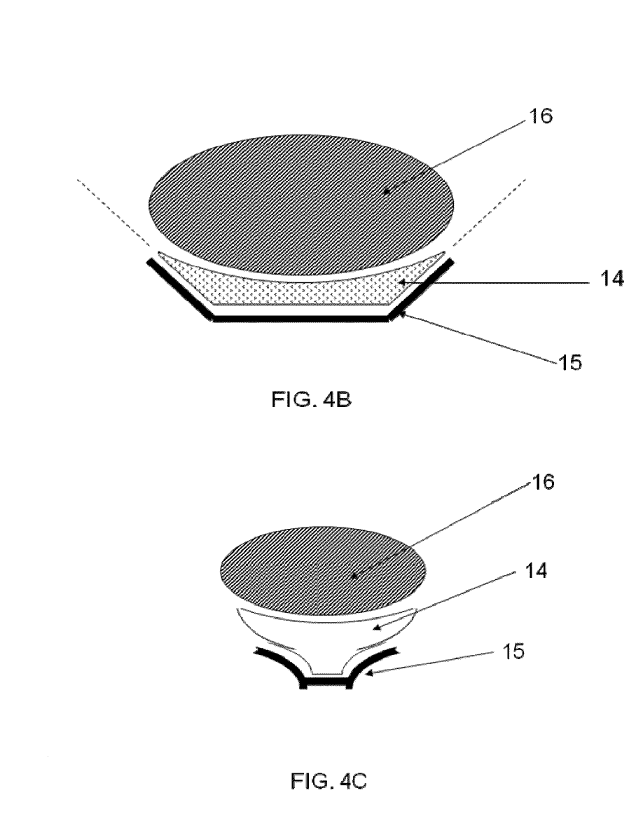

In some embodiments, the structure 14 may be in a form of an elongate shell. As viewed from a cross section, the elongate shell structure may have a top surface with a first concave curve, and a bottom surface with a second concave curve. In some embodiments, the structure 14 may be in a form of an elongate body with a cut-out region from the top. As such, the elongate body structure may have a curved top surface, a flat bottom surface, and side surfaces extending from the bottom to the top surfaces, as shown in FIG. 4A. This configuration assists coupling of the structure 14 to e.g. a couch 15. In some embodiments the side surfaces of the structure 14 and the couch 15 are slanted so that any artifact induced with the extended plane of the side surfaces (shown as dotted lines in FIG. 4B), lie outside the imaged volume of the patient or patient part 16. The side surfaces may be slanted to have an angle e.g. 100-175 degrees with the bottom surface. FIG. 4B shows a cross-section of the structure 14 having a generally trapezoidal shape with the top curved. In other embodiments the side surfaces of the structure 14 and couch 15 may also be curved to further eliminate plane induced artifacts due to either attenuation or scattering (FIG. 4C). The expanse of the planar bottom surface may be minimized. FIG. 4C shows a cross-section of the structure 14 having a generally bowl shape with the top curved. While structures with specific configurations are described, structures with other configurations are possible with reduced expanse of planar surfaces at least at the locations close to the patient or patient part 16.

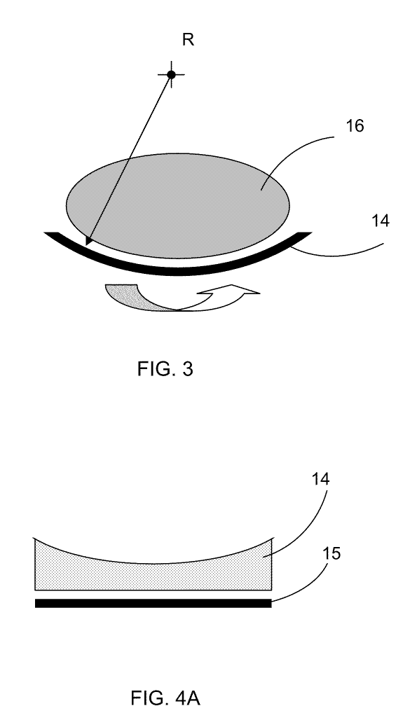

In some embodiments, the structure 14 may have a curved top surface with a substantially constant radius of curvature along the length of the structure, forming e.g., a semi-cylindrical shell structure, as shown in FIG. 3. The structure 14 may be rotated or tilted around the cylindrical axis of the structure 14 to replicate the same or close to the same patient-structure configuration each time the patient returns to the structure for treatment or treatment planning. Since the structure 14 is cylindrical in shape (as opposed to e.g. elliptical), the arc of contact with the patient boundary can be more easily reproduced. This is advantageous because for comparison of images and duplication of positioning on the same or different platforms, it is desirable that the patient's anatomy contacts an identical curved surface in as close to the same way as any earlier reference case. Alternatively, in some embodiments, the elongate structure 14 may have a top surface with varied radius of curvature along the length to accommodate different body portions, or provide patient comfort. The curved surface may be continuous or be constructed by a plurality of planar surfaces forming a generally curved geometry.

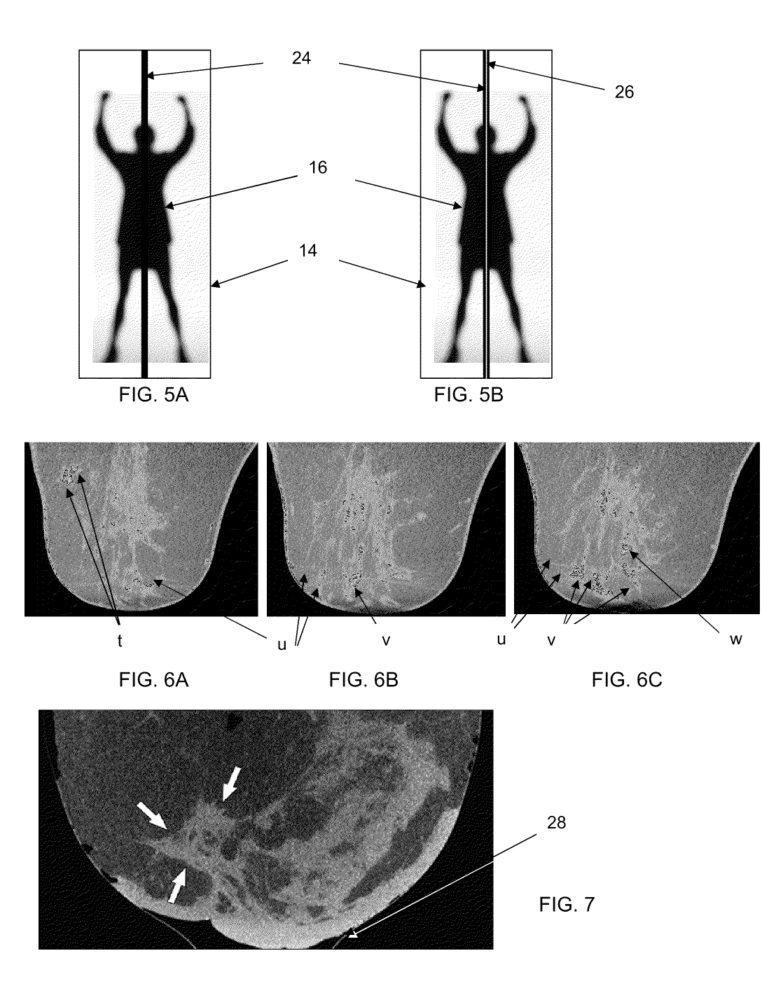

FIGS. 5A-5B illustrate exemplary embodiments of the structure 14 and methods of aligning e.g. a patient's body 16 to provide close patient realignment each time the patient returns to the structure for treatment, treatment planning, or other post diagnosis imaging tasks. In FIG. 5A, an alignment line 24 may be disposed along the length of an elongate body structure 14 or an elongate semi-cylindrical shell structure 14. The alignment line 24 may be centered on the symmetry axis of the curvature of the elongate structure 14. When in use, the patient 16 is instructed and/or assisted to first sit on the structure 14, then turn, and align the hips, legs or feet to be centered on the alignment line 24. Then the patient may carefully lie back centered over the alignment line 24, with technician assistance as needed. In the embodiment shown in FIG. 5B, an additional light line 26 such as a laser line (shown as a white line) may be projected over the top of the patient 16. The projected laser line 26 may superpose the alignment line 24 on the curved surface for close alignment of many patient body parts such as navel, sternum, throat, nose, eyes etc.

In conventional radiation therapy, couches with flat top surfaces have long been used for ease of patient alignment, identical cross sectional imaging for treatment planning and treatment delivery. However, flat top surface causes artifacts such as streak artifacts which create difficulties in viewing targets, especially for small cancer lesions. The imaging uncertainty or inaccuracy in turn hinders accurate treatment delivery and may expose the adjacent healthy tissue to unnecessary radiation doses. The resistance to curved treatment machine support surfaces may come in part from the repeated deliveries of therapeutic treatments in as many as 30 separate sessions over time periods up to 6 weeks in typical external beam radiotherapy. Many times the diagnosis and planning images have been taken using flat support even if they have higher artifact content. This is partly because there is less variation in the boundary shape and position and rotation of the patient body part interfacing with the support in such cases. Use of curved supports in such cases may cause the diagnosis and planning images less accurate in guiding the treatment delivery to the planned cancer lesions while sparing healthy regions. The invention provides for solutions to eliminate or at least mitigate artifacts by using structures with a curved top surface. By using alignment lines, the invention provides solutions for minimizing efforts to realign or reposition patients on a curved surface for treatment delivery or planning. Further, the apparatus of the invention may be used with dynamic adaptive treatment delivery system that may track, follow, correct and re-plan treatment delivery to adapt to any unavoidable motion, such as breathing, cardiac motion, and bowel and bladder or any other movement. With the capability of dynamic re-planning of each treatment session, the variable change of the curved support and the patient's body interfacing with this support can be accommodated for each treatment session.

The structure 14 may be an integral part of e.g., a couch or the like. Alternatively, the structure 14 may be removably coupled to and detachable from a couch. For instance, the structure 14 with a curved top surface may be coupled to a conventional couch which has a flat top. The structure 14 may include extensions or attachments etc. to couple the structure to a couch. Any suitable means such as removable pins, bolts, track or the like may be used to secure the structure 14 to a couch. In use, a patient or a body portion 16 may be positioned on the structure 14 in any suitable positions including but not limited to supine, prone, and leaning etc. The structure 14 may be adjustable or movable by e.g. rotating or tilting.

The structure 14 may be constructed with any suitable materials that transmit radiation such as X-ray radiation beam. Since the structure 14 would be positioned between radiation sources and image acquisition devices in use, it would be desirable that the structure 14 be constructed with materials that have low radiation attenuation coefficients. Suitable materials may include methacrylate plastics, carbon fiber composites, solid foams of various materials, or aerogels. In some embodiments such as shown in FIG. 4A-4C, the structure 14 may be a hollow structure. For example, the structure shown in FIGS. 4A, 4B and 4C may be constructed with a hollow interior, with its outer surface reinforced with a thin and strong material such as carbon fiber. Alternatively, the hollow interior may be optionally filled with light materials such as styrafoam.

In some embodiments, the structure 14 may include a member having a cavity with a curved surface. The cavity may be generally conformal to a shape of the body portion when pendulous. For example, the structure 14 may have a curved interior surface forming a generally cup-shaped cavity to receive a patient breast.

FIGS. 6A-6B are cone beam CT slice images of a breast cancer patient. The images were obtained from a patient laying in a prone position on a table, which had an opening to allow the breast to fall through. An X-ray source and an imager were supported on a C-arm structure and positioned opposite to each other. The X-ray source was configured to generate cone beams at KV energy levels. The imager was a flat panel KV imager. The breast was imaged as the C-arm structure carrying the X-ray source and imager rotated about a vertical axis around the breast. To make cancer lesions more obvious or identify small cancer legions, an iodine contrast agent was injected to the patient about 140 seconds before the CT data set was acquired in 16.6 seconds of X-ray exposure. The patient's breast was so pendulous that it would drop down out of the scan field of view if the breast was not held by any structure.

For comparison, a structure constructed with carbon fiber and having a flat surface was used to hold or support the breast at the bottom so that all the tissue was positioned within the scan field of view. Unfortunately this flat support introduced streak artifacts as shown at the bottom of the images in FIGS. 6A-6C.

In FIGS. 6A-6C, the green contour lines correspond to CT Hounsfield Unit (HU) values of 81 HU. This is one standard deviation above the mean of healthy, non cancerous, glandular tissue in this patient. The probability that a normal glandular distribution would have these green HU values is 16%. The blue contour lines correspond to HU values of 111 HU, which is 1.6 standard deviations above the healthy glandular mean with a 3.6% probability of occurring in a normal glandular distribution. The red contour lines correspond to HU values of 142 HU, which is 2.4 standard deviations above the healthy glandular mean with a probability of only 0.0047% of occurring in a normal glandular distribution. U.S. patent application Ser. No. 11/864,856 field Sep. 27, 2008 entitled “Cancer Detection, Diagnosis, Staging, Treatment Planning, Delivery and Monitoring Using X-Ray Attenuation Coefficient Distributions” discloses the use of X-ray attenuation coefficients distribution in cancer detection, diagnosis, treatment, and monitoring. U.S. patent application Ser. No. 11/864,856 is incorporated herein by reference in its entirety.

The red contour surfaces (t) shown in FIG. 6A correctly identify two cancer lesions in this patient that were biopsy confirmed to be invasive ductal carcinomas. Such red contour region identification of other suspect cancerous regions-of-interest (ROI) was applied to other slice images. Unfortunately the streak artifacts introduce HU error exceeding 50 HU. The introduced streak artifacts (u) confused the ROI (v) as shown in FIGS. 6B and 6C. In regions without artifacts such as near the middle of FIG. 6C, the low probability, red contour identification of true suspect cancerous ROI (w) is clearly shown.

FIG. 7 is a cone beam CT image of a cancer patient breast. This CT image was obtained from a patient breast supported by a structure 28 having a cup-shaped cavity. As shown in FIG. 7, a support structure 28 with a curved surface produces a CT slice image free of streak artifacts. The lesion indicated by the arrows in FIG. 7 is biopsy confirmed cancer lesion present in this patient's breast.

The structure 28 may be custom made to match the size and/or shape of the body portion such as a patient breast to provide a close fit. The structure 28 may be rigid. As used herein, the term “rigid” refers to a state of the structure that is not pliable by the body portion which is held by the structure. A rigid structure may provide physical support for the body portion, define the shape of the body portion, or immobilize or stabilize the body portion under treatment. The structure's interior surface may be lined with a thin soft layer such as silicone foams to increase comfort to the patient. U.S. patent application Ser. No. 12/105,795 filed Apr. 18, 2008 discloses a structure useful in radiation therapy and imaging of a patient's breast, the disclosure of which is incorporated herein by reference in its entirety.

The structure 28 may be used in treatment of a body part such as a breast of a patient in a prone position. Alternatively, the structure 28 may be used in treating a breast of a patient in a supine or other position. As such, one or more small holes may be provided in the wall of the structure 28 to connect the interior with a vacuum source. This permits the patient to be treated in the supine position yet with the breast held by the structure so that the breast is generally not compressed by gravity against the patient's rib cage. Instead, the breast may be held in a conformation very similar to the shape it would assume if the patient were prone and the gravity acted to pull the breast away from the chest wall. This increases patient comfort and improve treatment beam access compared to the prone position.

The exterior of the structure 28 can be in any suitable shape. By way of example, the structure 28 may have a hemispherical external shape generally matching the interior cavity shape of the structure. As such, the structure 28 may have an approximately constant wall thickness. The structure 28 may also have an external shape like a cylinder with an approximately hemispherical cavity within it. As such, the wall thickness of the structure varies. The material or materials made of the structure may be chosen to have approximately the same average beam absorption and/or scattering properties as the breast tissue so that the treatment beam transverse a rotation axis of the structure passes through an about constant length of materials (breast and structure). As such, the effective length of the structure-breast material that is traversed by the radiation beam at the nipple end of the breast is about the same as the length that is close to the chest wall. This advantageously aids in treatment planning.

In some embodiments, the structure 28 may include one or more markers (not shown) such as radio-opaque markers to aid in e.g. X-ray imaging. Optical markers may also be provided in the structure for a camera to accurately track breathing motion of the patient in real time. This allows compensation of patient's breathing motion by various means during treatment. For instance, the breathing motion can be compensated by controlling the radiation source. The radiation source may be controllably turned on or off at specified intervals, thus effectively “freezing” the treatment volume in position. By way of example, SmartTrack/RPM Respiratory Gating System available from Varian Medical Systems, Inc. in Palo Alto, Calif. may be employed in conjunction with the embodiments that include markers.

The structure 28 may include securing members to secure the structure to immobile structures such as to a couch, or to a shield protecting the patient's torso or thorax. The securing member may be connected to a support structure containing springs, gas struts, or similar passive devices, or an active servo system, to maintain a substantially constant supporting force on the securing structure while accommodating motion of the patient's chest wall due to breathing.

Returning to FIG. 2, the first and second radiation sources 12, 20, and the first and second image acquisition devices 18, 22 may be coupled to a computer system (not shown), which controls the operation of the radiation apparatus 10. The computer system receives, stores, and executes a treatment plan established in a pre-treatment planning session. The treatment plan may be established based on the nature, size, shape, and location of the target in the patient. The treatment plan may also include reference data regarding the position of the target, and the relationship between the target movement and the patient's inter- or intra-fraction movement established during a pre-treatment session for image-guided radiation therapy (IGRT). The reference data or the relationship data can be obtained by any suitable imaging techniques such as planar radiography, ultrasound (US), computed tomography (CT), single photon emission computed tomography (SPECT), magnetic resonance imaging (MRI), magnetic resonance spectroscopy (MRS), positron emission tomography (PET), etc.

The computer system also receives and stores digital signals from the first and second image acquisition devices 18, 22, and generates image data sets representing real time or near real time images of the target, from which the shape and location of the target can be visualized and determined in real or near real time. The real time image data are compared with the reference data obtained in the pre-treatment session. The results can then be used to control the first radiation source 12 to generate therapeutic radiation of a determined dose during the treatment session.

Exemplary embodiments of radiation apparatus and method have been described. Those skilled in the art will appreciate that various modifications may be made within the spirit and scope of the invention. For example, the structure may be incorporated into cancer treatment platforms with image guidance by e.g. X-ray CT including cone beam, helical, multislice, and single slice CT. Such cancer treatment platforms may include platforms using external radiation beams with C-arm gantry, enclosed ring gantries, and robotic gantries, and treatment platforms using one or more isotope sources. The treatment platforms may also include other energetic treatment modalities such as RF ablation, focused microwaves, ultrasound, and surgical treatment platforms such as robotic guided surgery. All these or other variations and modifications are contemplated by the inventors and within the scope of the invention.

What is claimed is:

1. A radiation apparatus, comprising:
a first radiation source configured to generate radiation suitable for therapeutic treatment; and
a structure comprising a curved surface adapted to receive a body portion to be treated during a therapeutic treatment.

2. The radiation apparatus of claim 1 further comprising a first image acquisition device operatively disposed opposite to the first radiation source.

3. The radiation apparatus of claim 1 further comprising a second radiation source configured to generate radiation suitable for diagnostic imaging, and a second image acquisition device operatively disposed opposite to the second radiation source.

4. The radiation apparatus of claim 1 wherein said structure comprises an elongate body having a first end and a second end, and a curved top surface extending from the first end to the second end.

5. The radiation apparatus of claim 4 further comprising a curved bottom surface extending from the first end to the second end.

6. The radiation apparatus of claim 4 further comprising a flat bottom surface extending from the first end to the second end.

7. The radiation apparatus of claim 6 further comprising a first and a second side surface each extending outwardly from said flat bottom surface to the curved top surface.

8. The radiation apparatus of claim 7 wherein said first and second side surfaces are curved.

9. The radiation apparatus of claim 4 wherein said curved surface has a substantially continuous radius of curvature.

10. The radiation apparatus of claim 4 wherein said curved surface has a substantially constant radius of curvature.

11. The radiation apparatus of claim 4 wherein said structure comprises an alignment line over the curved surface extending from the first end to the second end, said alignment line being substantially centered on a symmetry axis of the curvature of the structure.

12. The radiation apparatus of claim 4 further comprising a couch, and said structure is removably coupled to the couch.

13. The radiation apparatus of claim 12 wherein said structure has a hollow interior.

14. The radiation apparatus of claim 12 wherein said structure is movable relative to the couch.

15. The radiation apparatus of claim 1 wherein said structure has a curved surface forming a cup-shaped interior adapted to receive the body portion.

16. The radiation apparatus of claim 15 wherein said cup-shaped interior is generally conformal to a shape of the body portion when pendulous.

17. A radiation apparatus comprising:
a therapeutic radiation source, a first image acquisition device operatively disposed opposite to the therapeutic radiation source;
a diagnostic radiation source, a second image acquisition device operatively disposed opposite to the diagnostic radiation device; and
a structure comprising a curved surface adapted to receive a body portion to be irradiated by radiation beams from the therapeutic radiation source and the diagnostic radiation source.

18. The radiation apparatus of claim 17 wherein said structure comprises an elongate body having a first end, a second end, and a curved top surface extending from the first end to the second end and adapted to receive a patient in a laying position.

19. The radiation apparatus of claim 18 wherein said structure further comprising a curved bottom surface extending from the first end to the second end.

20. The radiation apparatus of claim 18 wherein said structure further comprising a flat bottom surface extending from the first end to the second end.

21. The radiation apparatus of claim 18 further comprising a couch, wherein said structure is removably coupled to the couch.

22. The radiation apparatus of claim 18 wherein said structure has a hollow interior.

23. The radiation apparatus of claim 18 wherein said structure comprises an alignment line over the curved surface extending from the first end to the second end, said alignment line being substantially centered on a symmetry axis of the curvature of the structure.

24. A method of irradiating a body portion of a patient, comprising the steps of:
placing a patient on a structure, said structure comprising an elongate body having a first end, a second end, and a curved top surface extending from the first end to the second end and adapted to receive the patient, said elongate body comprises an alignment line over the curved surface;
positioning the patient in a laying position with reference to the alignment line; and
delivering radiation to a target in the patient.

25. The method of claim 24, wherein the positioning step comprises aligning one or more anatomies of the patient with the alignment line.

26. The method of claim 24 wherein said one or more anatomies comprise navel, sternum, throat, nose, and eye.

27. The method of claim 24 further comprising the step of projecting a light line over the patient superposing the alignment line.
